(19) United States
(12) Patent Application Publication
(10) Pub. No.: US 2013/0231599 A1
(43) Pub. Date: Sep. 5, 2013

(54) ANTIMICROBIAL ADHESIVE FORMULATION AND FILM DRESSINGS HAVING AN ANTIMICROBIAL ADHESIVE

(71) Applicant: Patrick E. Eddy, Allendale, MI (US)

(72) Inventor: Patrick E. Eddy, Allendale, MI (US)

(21) Appl. No.: 13/780,500

(22) Filed: Feb. 28, 2013

Related U.S. Application Data
(60) Provisional application No. 61/605,302, filed on Mar. 1, 2012.

(51) Int. Cl. A61K 31/695; A61F 13/00; A61L 15/16

(52) U.S. Cl. CPC A61K 31/695; A61L 15/16; A61F 13/00063
USPC 602/48; 424/448; 514/63

(57) ABSTRACT

An adhesive formulation is provided for use in a film wound dressing. The adhesive formulation includes a medical-grade adhesive and an antimicrobial substance. This adhesive formulation may be coated onto one or more surfaces of a medical-grade transparent film suitable for a wound dressing. The antimicrobial material may be a silane quaternary ammonium salt. The silane quaternary ammonium salt may comprise 3-(trimethoxysilyl) propyldimethyloctadecyl ammonium chloride. The 3-(trimethoxysilyl) propyldimethyloctadecyl ammonium chloride may constitute about 72% of the silane quaternary ammonium salt. The medical-grade adhesive may comprise an acrylic adhesive. Further, the adhesive formulation may include about 0.01% to about 60% of the antimicrobial substance. The transparent film dressing may further include a release liner applied over the coated side(s) of the transparent film. The transparent film may comprise polyurethane.

### CROSS-REFERENCE TO RELATED APPLICATIONS

This application claims priority under 35 U.S.C. §119(e) upon U.S. Provisional Patent Application No. 61/605,302, entitled “ANTIMICROBIAL ADHESIVE FORMULATION AND FILM DRESSINGS HAVING AN ANTIMICROBIAL ADHESIVE” filed on Mar. 1, 2012, by Patrick E. Eddy, the entire disclosure of which is incorporated herein by reference.

### BACKGROUND OF THE INVENTION

The embodiments described herein generally relate to an adhesive formulation for a film dressing, and to an adhesive-coated film dressing.

### SUMMARY OF THE INVENTION

According to an embodiment of the present invention, a film dressing is provided that comprises: a film having a first side and a second side; an adhesive formulation coated on at least one of the sides of the film; and a release liner applied over the adhesive formulation. The adhesive formulation comprises a mixture of a medical-grade adhesive and an antimicrobial substance, and includes about 0.01% to about 60% of the antimicrobial substance. The antimicrobial substance is a silane quaternary ammonium salt comprising 3-(trimethoxysilyl) propyldimethyloctadecyl ammonium chloride. The medical-grade adhesive comprises an acrylic adhesive. The film wound dressing may be transparent or opaque.

According to another embodiment of the present invention, a transparent film dressing is provided that comprises a transparent film having an adhesive formulation coated on one or more sides, where the adhesive formulation comprises a medical-grade adhesive and an antimicrobial substance. The transparent film dressing may further include a release liner applied over the coated side(s) of the transparent film. The transparent film may comprise polyurethane.

According to another embodiment of the present invention, an adhesive formulation is provided for use on a film wound dressing, the adhesive formulation comprising: a medical-grade adhesive and an antimicrobial substance.

These and other features, advantages, and objects of the present invention will be further understood and appreciated by those skilled in the art by reference to the following specification, claims, and appended drawings.

### BRIEF DESCRIPTION OF THE DRAWINGS

In the drawings:

FIG. 1 is perspective view of a film dressing constructed in accordance with an embodiment of the present invention;

FIG. 2 is a schematic representation of a monomer that may be used in the embodiments described herein as an antimicrobial treatment substance;

FIG. 3 is a schematic representation of a plurality of the monomers shown in FIG. 2 as applied to a treated surface;

FIG. 4A is a schematic representation of the monomer shown in FIGS. 2 and 3 illustrating a first step in the manner by which the monomer destroys a microbe;

FIG. 4B is a schematic representation of the monomer shown in FIGS. 2 and 3 illustrating a second step in the manner by which the monomer destroys a microbe; and

FIG. 4C is a schematic representation of the monomer shown in FIGS. 2 and 3 illustrating a third step in the manner by which the monomer destroys a microbe.

### DETAILED DESCRIPTION OF PREFERRED EMBODIMENTS

Reference will now be made in detail to the present preferred embodiments of the invention, examples of which are illustrated in the accompanying drawings. Wherever possible, the same reference numerals will be used throughout the drawings to refer to the same or like parts. In the drawings, the depicted structural elements are not to scale and certain components are enlarged relative to the other components for purposes of emphasis and understanding.

A novel adhesive formulation for film dressings is disclosed herein that not only provides the requisite adhesion properties, but also eliminates bacteria on contact. In general, the adhesive formulation comprises an adhesive and an antimicrobial substance, such as a silane quaternary ammonium salt. The adhesive may be a medical-grade acrylic based or synthetic rubber based. Preferred silane quaternary ammonium salts are MicrobeCare™ XLP, which is available from MicrobeCare, LLC of Allendale, Mich., and AEM 5772 Antimicrobial, which is available from Aegis Environments of Midland, Mich. These particular antimicrobial substances include an active ingredient of about 72% 3-(trimethoxysilyl) propyldimethyloctadecyl ammonium chloride and about 28% inert ingredients. The antimicrobial could also be AEGIS Microbe Shield™ (from Aegis Environments, Midland, Mich.), which is a copolymer of chloropropyltrihydroxysilane and octadecylaminodimethyltrihydroxysilylpropyl ammonium chloride.

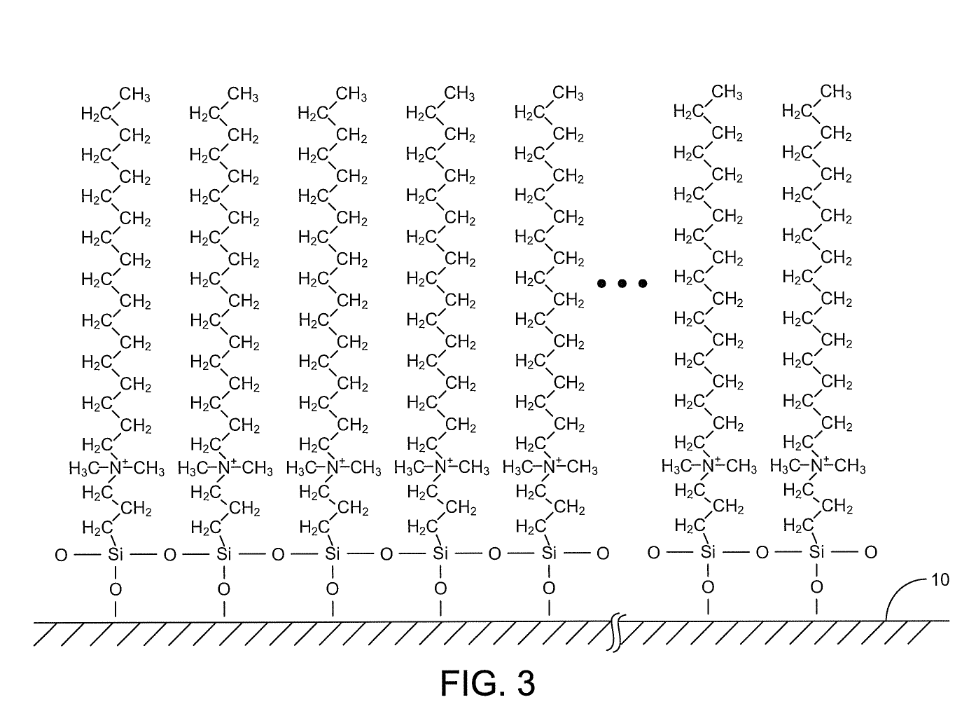

Silane quaternary ammonium salts are particularly well suited for the antimicrobial material as they are long lasting and are capable of emitting ions that aid in the destruction of a microbe. FIG. 2 shows a schematic representation of a monomer form 30 of a preferred organofunctional silane. As illustrated, monomer 30 includes a silane base 31 for bonding to a surface, a positively charged nitrogen molecule 34, and a long molecular chain 32. As shown in FIG. 3, the silane bases of these monomers covalently and permanently bond to each other and to any surfaces 10 in such a way that the long molecular chains are aligned and pointing outward from such surfaces 10. This tight bonding provides a micropolymer network that serves as a protective coating on the outside of the surface 10 that destroys any microbes that come into contact. In addition, silane quaternary ammonium salts are preferable as they are substantially free from arsenic, silver, tin, heavy metals and polychlorinated phenols. Also, they are safe to apply to the skin or to a wound of a patient.

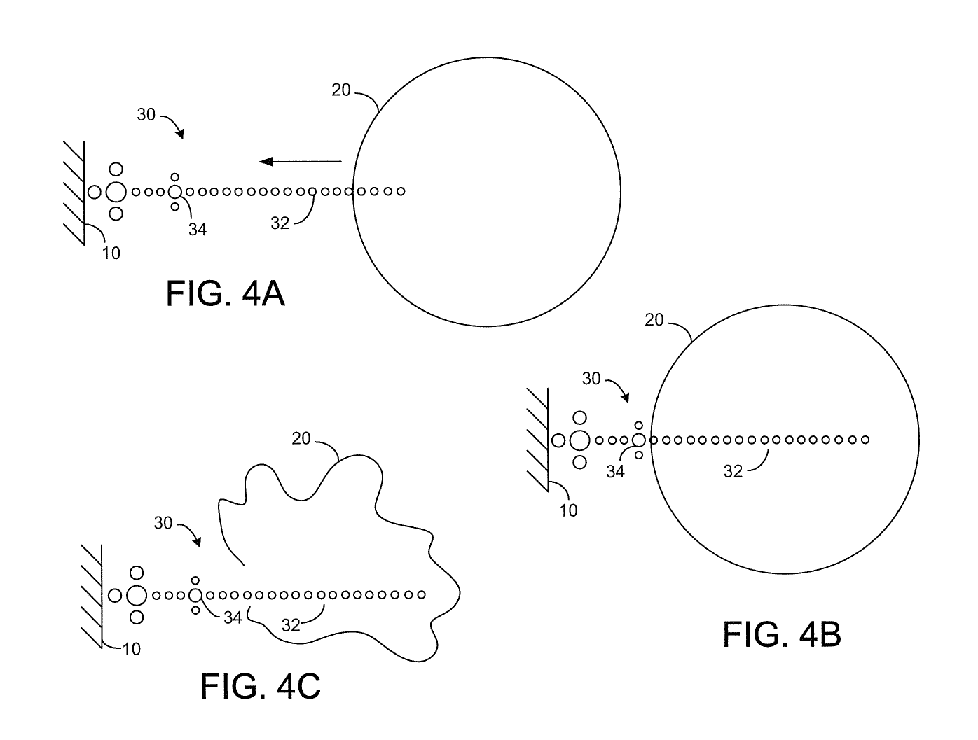

The manner by which the preferred organofunctional silane destroys microbes is illustrated in FIGS. 4A-4C. Microbes may include bacteria, mold, mildew, algae, etc. As shown in FIG. 4A, the cell membrane 20 of the microbes are attracted to, and then are punctured by, the long molecular chains 32 of the monomers 30. As the microbes are drawn closer because of the positive-negative ion exchanges, the monomers 30 penetrate further into the cell membranes 20 as shown in FIG. 4B. Once the cell membranes 20 are penetrated deeply, they are physically ruptured by a sword-like action and then electrocuted by positively charged nitrogen molecules 34 of the monomers 30, thus destroying the microbes as illustrated in FIG. 4C. Thus, the microbes are eliminated without “using up” any of the antimicrobial active ingredients, which remain in the adhesive formulation ready to continue protecting the patient against further microbial contamination.

The preferred organofunctional silane also prevents odor, staining and product deterioration that may be associated with microbe contamination. The preferred organofunctional silane is also beneficial because it covers a broad spectrum of activity with no negative effects or drawbacks and is easily incorporated and easily verifiable.

The antimicrobial substance may be mixed into the adhesive in various amounts of anywhere from about 0.01% to about 60% to achieve the desired degree of antimicrobial activity while not compromising the adhesion properties of the resulting mixture.

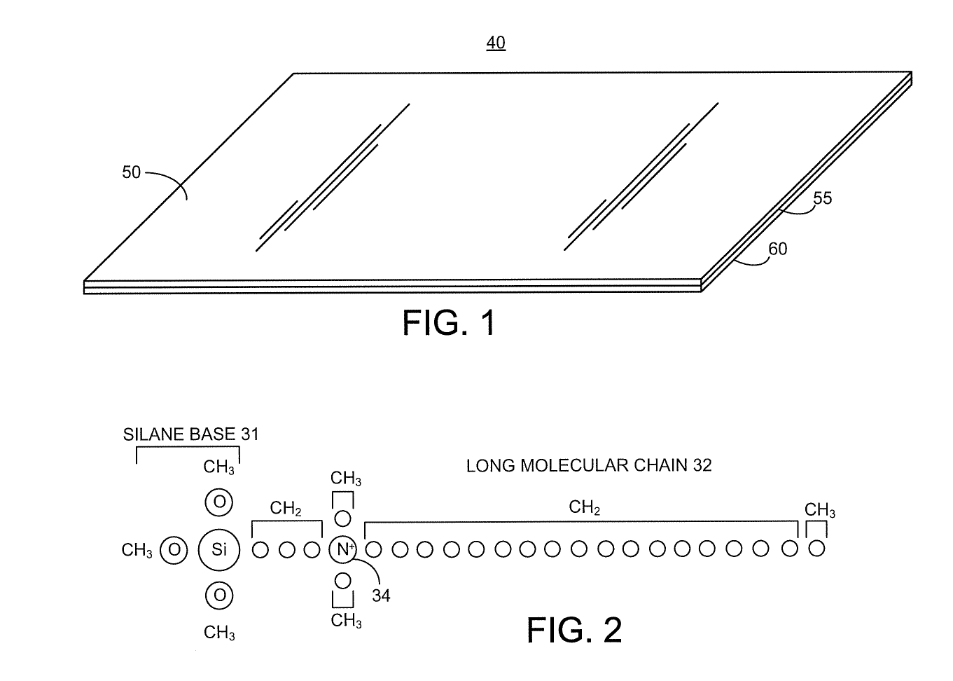

The adhesive formulation 55 (FIG. 1) is then coated on one side or both sides of a medical-grade film 50, which may be a transparent film of the type commonly used for a wound dressing 40. One suitable transparent film is a 1-mil polyurethane film available from DermaMed Coatings Company, LLC of Tallmadge, Ohio. The coating method may be a knife-over-roll method. A release liner 60 may be applied to the coated side(s) of the transparent film 50.

The transparent film 50 may be used as a wound dressing or for any other medical use such as securing an intravenous needle to a patient.

The film 50 may also be opaque and may be of the type that is commonly used for bandages or medical tape.

The above description is considered that of the preferred embodiments only. Modifications of the invention will occur to those skilled in the art and to those who make or use the invention. Therefore, it is understood that the embodiments described above are merely for illustrative purposes and not intended to limit the scope of the invention, which will be defined by claims as interpreted according to the principles of patent law, including the doctrine of equivalents.

What is claimed is:

1. A film dressing comprising:
a film having a first side and a second side;
an adhesive formulation coated on at least one of said sides of said film; and
a release liner applied over said adhesive formulation,
wherein said adhesive formulation comprises a mixture of a medical-grade adhesive and an antimicrobial substance,
wherein said adhesive formulation includes about 0.01% to about 60% of said antimicrobial substance,
wherein said antimicrobial substance is a silane quaternary ammonium salt comprising 3-(trimethoxysilyl) propyldimethyloctadecyl ammonium chloride, and
wherein said medical-grade adhesive comprises an acrylic adhesive.

2. The film dressing of claim 1, wherein said film comprises polyurethane.

3. The film dressing of claim 1, wherein said film is transparent.

4. The film dressing of claim 1, wherein said film is opaque.

5. The film dressing of claim 1, wherein said 3-(trimethoxysilyl) propyldimethyloctadecyl ammonium chloride constitutes about 72% of said silane quaternary ammonium salt.

6. A transparent film dressing comprising:
a transparent film having an adhesive formulation coated on one or more sides,
wherein said adhesive formulation comprises a mixture of a medical-grade adhesive and an antimicrobial substance.

7. The transparent film dressing of claim 6, wherein said transparent film dressing further includes a release liner applied over the coated side(s) of said transparent film.

8. The transparent film dressing of claim 6, wherein said transparent film comprises polyurethane.

9. The transparent film dressing of claim 6, wherein said antimicrobial substance is a silane quaternary ammonium salt.

10. The transparent film dressing of claim 9, wherein said silane quaternary ammonium salt comprises 3-(trimethoxysilyl) propyldimethyloctadecyl ammonium chloride.

11. The transparent film dressing of claim 10, wherein said 3-(trimethoxysilyl) propyldimethyloctadecyl ammonium chloride constitutes about 72% of said silane quaternary ammonium salt.

12. The transparent film dressing of claim 11, wherein said medical-grade adhesive comprises an acrylic adhesive.

13. The transparent film dressing of claim 6, wherein said medical-grade adhesive comprises an acrylic adhesive.

14. The transparent film dressing of claim 6, wherein said adhesive formulation includes about 0.01% to about 60% of said antimicrobial substance.

15. An adhesive formulation for use on a film wound dressing, the adhesive formulation comprising:
a mixture of a medical-grade adhesive and an antimicrobial substance.

16. The adhesive formulation of claim 15, wherein said antimicrobial substance is a silane quaternary ammonium salt.

17. The adhesive formulation of claim 16, wherein said silane quaternary ammonium salt comprises 3-(trimethoxysilyl) propyldimethyloctadecyl ammonium chloride.

18. The adhesive formulation of claim 17, wherein said 3-(trimethoxysilyl) propyldimethyloctadecyl ammonium chloride constitutes about 72% of said silane quaternary ammonium salt.

19. The adhesive formulation of claim 18, wherein said medical-grade adhesive comprises an acrylic adhesive.

20. The adhesive formulation of claim 15, wherein said medical-grade adhesive comprises an acrylic adhesive.

21. The adhesive formulation of claim 15, wherein said adhesive formulation includes about 0.01% to about 60% of said antimicrobial substance.
